(19) United States
(12) Patent Application Publication
(10) Pub. No.: US 2025/0079014 A1
(43) Pub. Date: Mar. 6, 2025

(54) REHABILITATION MENU DETERMINATION ASSISTANCE APPARATUS, SYSTEM, METHOD, AND NON-TRANSITORY COMPUTER-READABLE MEDIUM STORING PROGRAM THEREFOR

(71) Applicant: NEC Corporation, Minato-ku, Tokyo (JP)

(72) Inventors: Yusuke Ito, Tokyo (JP); Yuki Kosaka, Tokyo (JP); Shigemi Kitahara, Tokyo (JP)

(73) Assignee: NEC Corporation, Minato-ku, Tokyo (JP)

(21) Appl. No.: 18/723,948

(22) PCT Filed: Dec. 20, 2022
(86) PCT No.: PCT/JP2022/046972

§ 371 Date: Jun. 25, 2024

(30) Foreign Application Priority Data
Jan. 26, 2022 (JP) 2022-010530

(51) Int. Cl. G16H 50/30; G16H 50/70

(52) U.S. Cl. CPC G16H 50/30; G16H 50/70

(57) ABSTRACT

In an apparatus in the related art, there is a problem that a rehabilitation menu presented by a rehabilitation planning apparatus or the like is not utilized. A rehabilitation menu determination assistance apparatus according to an example embodiment includes: patient information acquisition means for acquiring reference patients having individual information from past information; similarity score calculation means for calculating a similarity score indicating similarity of individual information between an input reference patient and the reference patients; similarity ranking determination means for ranking the reference patients in a descending order of the similarity score; and first rearrangement processing means for rearranging ranks of the reference patients so that the reference patients including more rehabilitation menus commonly performed for a plurality of the reference patients among rehabilitation menus operated on the reference patients are ranked higher.

### TECHNICAL FIELD

The present invention relates to a rehabilitation menu determination assistance apparatus, a system, a method, and a non-transitory computer-readable medium storing a program thereof, and for example, relates to a rehabilitation menu determination assistance apparatus that presents a user with a rehabilitation menu that is operated on a past patient as a reference material, a system, a method, and a non-transitory computer-readable medium storing a program thereof.

### BACKGROUND ART

Rehabilitation (hereinafter, also referred to as “rehabilitation”) is carried out on the basis of a rehabilitation menu planned by a physical therapist, but it is known that a recovery speed of a patient varies depending on the degree of experience of the physical therapist who makes the plan. Therefore, Patent Literature I discloses a technique for formulating an optimal rehabilitation plan regardless of the experience of a physical therapist.

A rehabilitation planning apparatus described in Patent Literature 1 includes a similarity calculation unit that calculates similarity between a plurality of pieces of past information that is information about a plurality of past patients who have undergone rehabilitation in the past, and target patient information that is information about a target patient, a ranking unit that extracts one or more pieces of past information from the plurality of pieces of past information on the basis of the similarity and ranks the one or more pieces of past information in accordance with a predetermined ranking rule, and a determination unit that determines a rehabilitation history of the past patient which is associated with the past information as a rehabilitation plan for the target patient in accordance with a result of ranking of the past information.

### CITATION LIST

### Patent Literature

Patent Literature 1: Japanese Unexamined Patent Application Publication No. 2021-60769

### SUMMARY OF INVENTION

However, there is a problem that even though a rehabilitation planning apparatus or the like presents a rehabilitation menu that is determined to be optimal, a physical therapist does not know an effect obtained by operating the rehabilitation menu, and the rehabilitation menu presented by the rehabilitation planning apparatus or the like is not utilized such as investigation of a past rehabilitation history of the patient.

According to an example embodiment, there is provided a rehabilitation menu determination assistance apparatus including: a patient information acquisition unit configured to acquire reference patients having individual information from past information; a similarity score calculation unit configured to calculate a similarity score indicating similarity of individual information between an input target patient and the reference patients; a similarity ranking determination unit configured to rank the reference patients in a descending order of the similarity score; and a first rearrangement processing unit configured to rearrange ranks of the reference patients so that the reference patients including more rehabilitation menus commonly performed for a plurality of the reference patients among rehabilitation menus operated on the reference patients are ranked higher.

According to another example embodiment, there is provided a rehabilitation menu determination assistance system including: a rehabilitation menu determination assistance apparatus; and a terminal apparatus, wherein the rehabilitation menu determination assistance apparatus includes a patient information acquisition unit configured to acquire reference patients having individual information from past information; a similarity score calculation unit configured to calculate a similarity score indicating similarity of the individual information between an input target patient and the reference patients; a similarity ranking determination unit configured to rank the reference patients in a descending order of the similarity score; a first rearrangement processing unit configured to perform rearrangement so that the reference patients including more rehabilitation menus commonly performed on a plurality of the reference patients among the rehabilitation menus operated on the reference patients are ranked higher; and an output control unit configured to output information about the reference patients rearranged by the first rearrangement processing unit to the terminal apparatus.

According to still another example embodiment, there is provided a rehabilitation menu determination assistance method executed by arithmetic processing of a computer system including a storage device storing past information, including: patient information acquisition processing of acquiring reference patients having individual information from past information; similarity score calculation processing of calculating a similarity score indicating similarity of individual information between an input reference patient and the reference patients; similarity ranking determination of ranking the reference patients in a descending order of the similarity score; and first rearrangement processing of rearranging ranks of the reference patients so that the reference patients including more rehabilitation menus commonly performed for a plurality of the reference patients among rehabilitation menus operated on the reference patients are ranked higher.

According still another example embodiment, there is provided a non-transitory computer-readable medium storing a rehabilitation menu determination assistance program causing a computer to execute: patient information acquisition processing of acquiring reference patients having individual information from past information; similarity score calculation processing of calculating a similarity score indicating similarity of individual information between an input target patient and the reference patients; similarity ranking determination processing of ranking the reference patients in a descending order of the similarity score; and first rearrangement processing of rearranging ranks of the reference patients so that the reference patients including more rehabilitation menus commonly performed for a plurality of the reference patients among rehabilitation menus operated on the reference patients are ranked higher.

According to the example embodiments, it is possible to present information of a rehabilitation menu that can be easily utilized by a user for work.

### BRIEF DESCRIPTION OF DRAWINGS

FIG. 1 is a block diagram illustrating an example of a rehabilitation menu determination assistance apparatus according to an outline of a first example embodiment.

FIG. 2 is a block diagram illustrating an example of a configuration of a rehabilitation menu determination assistance system according to the first example embodiment.

FIG. 3 is a schematic view illustrating an example of a user interface screen displayed on a terminal apparatus in the rehabilitation menu determination assistance system according to the first example embodiment.

FIG. 4 is a schematic view illustrating an example of a hardware configuration of the rehabilitation menu determination assistance apparatus according to the first example embodiment.

FIG. 5 is a flowchart illustrating an example of an operation of the rehabilitation menu determination assistance apparatus according to the first example embodiment.

FIG. 6 is a block diagram illustrating an example of a rehabilitation menu determination assistance apparatus according to an outline of a second example embodiment.

FIG. 7 is a block diagram illustrating an example of a configuration of a rehabilitation menu determination assistance system according to the second example embodiment.

FIG. 8 is a schematic view illustrating an example of a user interface screen displayed on a terminal apparatus in the rehabilitation menu determination assistance system according to the second example embodiment.

FIG. 9 is a flowchart illustrating an example of an operation of the rehabilitation menu determination assistance apparatus according to the second example embodiment.

### EXAMPLE EMBODIMENT

### Outline of Example Embodiment

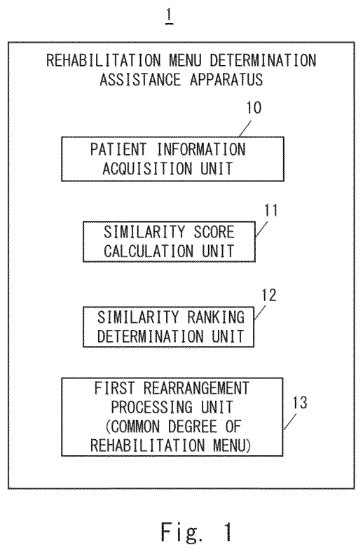

Prior to detailed description of an example embodiment, an outline of the example embodiment will be described. FIG. 1 is a block diagram illustrating an example of a rehabilitation menu determination assistance apparatus 1 according to an outline of a first example embodiment. As illustrated in FIG. 1, a rehabilitation menu determination assistance apparatus 1 includes a patient information acquisition unit 10, a similarity score calculation unit 11, a similarity ranking determination unit 12, and a first rearrangement processing unit 13.

The patient information acquisition unit 10 acquires a reference patient having individual information from past information. More specifically, the patient information acquisition unit 10 acquires the individual information of the reference patient in order to use the individual information as a reference for a rehabilitation menu to be operated on the input target patient. Here, the target patient is a patient who is scheduled to be rehabilitated by a physical therapist/occupational therapist (for example, a user). The reference patient is a patient different from the target patient, and is a patient who has been rehabilitated in the past.

The past information is a set of information about the reference patient, more specifically, a set of information indicating individual information about the reference patient. The past information may include information about individual information of the target patient. In addition, in the rehabilitation menu determination assistance apparatus 1 according to the first example embodiment, the information relating to the target patient is given from the user or is given when the user confirms the information with reference to the past information. Note that, items of the reference patient (the type of the individual information included in the information of the reference patient) and items of the information relating to the target patient (the type of the individual information included in the information of the target patient) are at least partially correspond to each other. Therefore, for the reference patient and the target patient, it is possible to compare corresponding items. In addition, the past information is associated with a rehabilitation history of the reference patient.

The similarity score calculation unit 11 calculates a similarity score indicating the similarity of the individual information between the input target patient and the reference patient. More specifically, the similarity score calculation unit 11 calculates a similarity score between the reference patient and the target patient for at least one of an age, a sex, a symptom, a history of an ability value during a rehabilitation period, and a goal of the ability value acquired by rehabilitation. A method of calculating the similarity score in the similarity score calculation unit 11 is determined in advance by a system designer.

In addition, the individual information of the target patient and the reference patient may include information of the physical therapist/occupational therapist in charge, and the similarity score may include a value related to the physical therapist/occupational therapist. In this case, it is conceivable to set the score such that the scores of physical therapists and occupational therapists who have longer years of experience than those of physical therapists and occupational therapists in charge of the target patient are higher.

The similarity ranking determination unit 12 ranks reference patients in a descending order of similarity scores. The first rearrangement processing unit 13 rearranges the rank of the reference patients so that the reference patients including more rehabilitation menus commonly operated on a plurality of reference patients among the rehabilitation menus performed on the reference patients are ranked higher.

More specifically, the first rearrangement processing unit 13 extracts, as a common rehabilitation menu, the rehabilitation menus operated on at least two reference patients for the reference patients arranged in the rank determined by the similarity ranking determination unit 12, and rearranges the orders of the reference patients so that the reference patients including more common rehabilitation menus are ranked higher.

As described above, according to the rehabilitation menu determination assistance apparatus 1, among reference patients having individual information close to the individual information of the target patient, it is possible to provide a method of presenting information so that a reference patient including a plurality of rehabilitation menus commonly operated on a plurality of reference patients is displayed higher. By presenting the rehabilitation information of the reference patient to a user by such display, the user can easily determine what kind of patient treatment was performed on a predetermined rehabilitation menu and what kind of effect was obtained. That is, by using the rehabilitation menu determination assistance apparatus 1, for example, it is easy to use a rehabilitation menu that is determined to be optimal and presented by a rehabilitation planning apparatus or the like.

### First Example Embodiment

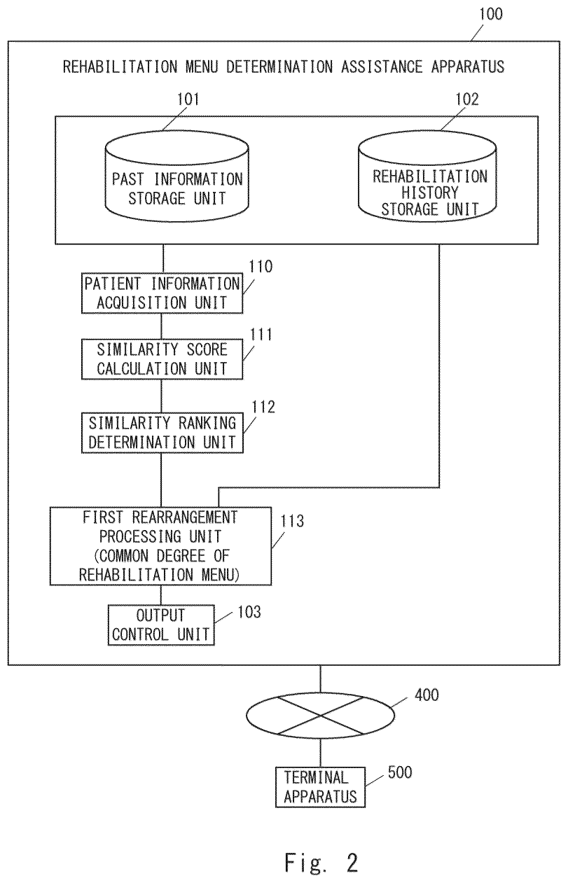

Hereinafter, an example embodiment of the present invention will be described with reference to the drawings. FIG. 2 is a block diagram illustrating an example of a configuration of a rehabilitation menu determination assistance system according to a first example embodiment. The rehabilitation menu determination assistance system according to the first example embodiment includes a rehabilitation menu determination assistance apparatus 100 and a terminal apparatus 500 which are communicably connected in a wired or wireless manner via a network 400.

The rehabilitation menu determination assistance apparatus 100 is configured as, for example, a server. In addition, the terminal apparatus 500 is any terminal such as a personal computer, a tablet terminal, or a smartphone. The terminal apparatus 500 includes an input apparatus and an output apparatus, and can input information to be transmitted to the rehabilitation menu determination assistance apparatus 100 and output (display) information received from the rehabilitation menu determination assistance apparatus 100. Although only one terminal apparatus 500 is illustrated in FIG. 2, the rehabilitation menu determination assistance system may include a plurality of the terminal apparatuses 500.

As illustrated in FIG. 2, the rehabilitation menu determination assistance apparatus 100 includes a past information storage unit 101, a rehabilitation history storage unit 102, an output control unit 103, a patient information acquisition unit 110, a similarity score calculation unit 111, a similarity ranking determination unit 112, and a first rearrangement processing unit 113.

The rehabilitation menu determination assistance apparatus 100 is an apparatus that rearranges and presents a rehabilitation menu operated on a reference patient similar to the target patient so that a user can easily utilize the rehabilitation menu for work. Here, in the following description, the rehabilitation menu includes at least one rehabilitation instruction to be operated on the target patient, and may be referred to as a rehabilitation program.

The past information storage unit 101 stores past information for each reference patient. Information of each item in the past information and information of each item of the target patient to be described later are, for example, numerically coded. Note that, since there is a high possibility that the past information is unlikely to be useful as a reference for rehabilitation of the target patient, it is preferable that the past information used for processing of the rehabilitation menu determination assistance apparatus 100 does not include past information relating to a specific patient (for example, patients with special circumstances).

The rehabilitation history storage unit 102 stores a history of rehabilitation for each reference patient. The past information stored in the past information storage unit 101 and the rehabilitation history stored in the rehabilitation history storage unit 102 are associated with each other for each reference patient.

Note that, in the example illustrated in FIG. 2, the past information storage unit 101 and the rehabilitation history storage unit 102 are illustrated separately, but these may be realized as one storage unit. Furthermore, in the present example embodiment, the rehabilitation menu determination assistance apparatus 100 includes the past information storage unit 101 and the rehabilitation history storage unit 102, but the past information storage unit 101 and the rehabilitation history storage unit 102 may be realized by an external apparatus. In this case, the rehabilitation menu determination assistance apparatus 100 may acquire the past information and the rehabilitation history from the external apparatus.

In the present example embodiment, the past information includes patient attributes, disease names, symptoms, ability values, personal goals, and the like. However, these are examples, and the past information is not limited thereto. The patient attributes specifically include, for example, any attribute information such as an age, a sex, and social information of the patient. Here, the social information is information indicating a social state of the patient, and includes a family structure, the presence or absence of a roommate, a place of residence, a type of a house building (whether the building is an apartment, a two-story detached building, or the like), information of a medical insurance of the patient, information of a nursing care insurance of the patient, and the like.

The ability value is an ability value for living activities of the patient, and is, for example, an ability value for activities of daily living (ADL) or instrumental activities of daily living (IADL). In the present example embodiment, the ability value included in the past information is specifically an evaluation point for each evaluation item in a function independence measure (FIM), but other ability values may be used.

In the present example embodiment, the ability value included in the past information is information (time series data) indicating the transition of the evaluation value for each evaluation item of the FIM. That is, the past information includes a history of ability values. In the present example embodiment, the history of the ability value includes a history of an ability value during rehabilitation (recovery rehabilitation) in a recovery rehabilitation hospital. This includes the ability value of the past patient before and after the execution of the rehabilitation indicated in the rehabilitation history associated with the past information (the rehabilitation history stored in the rehabilitation history storage unit 102). In addition, in the present example embodiment, the past information includes not only the history of the ability value at the time of the recovery rehabilitation but also the history of the ability value at the time of the rehabilitation in an acute phase hospital (acute phase rehabilitation) as the history of the ability value. In the present example embodiment, the ability value is a value for each of a plurality of types of abilities (a value for each item of the FIM), but may be an ability value for one type of ability.

The individual target is information indicating a target of an individual patient in rehabilitation. For example, the personal goal is as follows, but is not limited thereto.
“You can cross the road within the time when the traffic light is green”
“You can walk fast”
“Return to clerical work”
“One can live alone without nursing care”
“Score for each classification item of the ADL related index such as FIM is better than a predetermined value”
“Level of assistance required or a level of care required in a nursing field is better than a predetermined value”
“Returning to clerical work mainly including operation of a calculator on a desk can be performed”
“There is no need for assistance other than bathing, and if a specific care service is received, the resident can stay alone at home”
“From the perspective of the people around you, you can walk while avoiding obstacles without feeling uncomfortable”
“It is possible to go up and down stairs at home with light baggage”

In the present example embodiment, specifically, the rehabilitation history storage unit 102 stores a history of rehabilitation in a recovery rehabilitation hospital as a history of rehabilitation. The rehabilitation history is, for example, time-series data relating to rehabilitation contents for each predetermined period (for example, every week). The rehabilitation contents include, for example, a task to be achieved by rehabilitation and practice contents (programs) for achieving the task. Note that, a higher-level task and a lower-level task may be set as the tasks. In addition, the rehabilitation history may include time when an outing event occurs. Here, the outing event refers to a predetermined event accompanied by an outing such as staying out overnight or temporarily returning home and performing rehabilitation at home. In addition, the rehabilitation history may include various types of information such as identification information of a therapist who has performed an operation and a rehabilitation execution place.

The patient information acquisition unit 110 corresponds to the patient information acquisition unit 10 in FIG. 1, and acquires information of the target patient from the past information storage unit 101. More specifically, the patient information acquisition unit 110 acquires the information of the reference patients from the past information storage unit 101 in order to use the information as a reference for a rehabilitation menu to be operated on the input target patient from the terminal apparatus 500. For example, the user inputs the target patient information to the terminal apparatus 500, and the terminal apparatus 500 inputs the input target patient information to the rehabilitation menu determination assistance apparatus 100. Then, the target patient information received by the rehabilitation menu determination assistance apparatus 100 is input to the patient information acquisition unit 110.

In the present example embodiment, information of the same type as the past information is input to the similarity score calculation unit 111 as the target patient information. That is, in the present example embodiment, the target patient information includes patient attributes, disease names, symptoms, ability values, personal goals, and the like similarly to the past information. However, these are examples, and the target patient information is not limited thereto. Note that, the ability value of the target patient information is, for example, information (time series data) indicating transition of an evaluation value for each evaluation item of the FIM. In this manner, the target patient information includes a history of ability values. In the present example embodiment, the history of ability value in the target patient information includes at least a history of ability value during rehabilitation (acute rehabilitation) in an acute phase hospital. However, in a case where the target patient has already undergone rehabilitation (recovery rehabilitation) in the recovery rehabilitation hospital, a history of ability value at the time of rehabilitation (recovery rehabilitation) in the recovery rehabilitation hospital may be further included.

The similarity score calculation unit 111 corresponds to the similarity score calculation unit 11 in FIG. 1, and calculates a similarity score indicating the similarity of the individual information between the input target patient and the reference patient. More specifically, the similarity score calculation unit 111 may calculate a similarity score between the target patient information and the reference patient information by calculating a distance between a vector corresponding to the target patient information and a vector corresponding to the reference patient information. In this case, the smaller the distance, the higher the similarity score. Note that, the distance may be calculated by performing predetermined weighting for each element of the vector (that is, for each item of the reference patient information and the target patient information,). However, the above-described calculation method is an example, and any other calculation method that determines the similarity of characteristics between the target patient and the reference patient may be used. Note that, in a case where the information of the input target patient is only some items in the reference patient information, the similarity score calculation unit 111 may compare the target patient information with the reference patient information only for the items.

Here, in particular, in the present example embodiment, the information about the reference patient and the target patient includes a history of ability value for a predetermined rehabilitation period (specifically, the acute phase), and the similarity score calculation unit 111 calculates a similarity of the history of ability value for at least the predetermined rehabilitation period. More specifically, the similarity score calculation unit 111 calculates the similarity of a change pattern of the ability value in the predetermined rehabilitation period. Patients having a similar history of ability value (change patterns) tend to show similar recovery when performing the same rehabilitation content. Therefore, by considering the similarity of the history (change pattern) of the ability value for rehabilitation, the similarity between the past patient and the target patient can be more accurately determined.

Furthermore, in the present example embodiment, the information about the reference patient and the target patient includes a goal in rehabilitation (individual goal), and the similarity score calculation unit 111 calculates at least the similarity of the goal. Patients with similar goals in rehabilitation tend to show similar recovery when performing the same rehabilitation contents. Therefore, by considering the similarity of the goal in rehabilitation, the similarity between the reference patient and the target patient can be more accurately determined.

Note that, in the present example embodiment, as described above, the similarity score calculation unit 111 determines the similarity of the history of the ability value in a predetermined rehabilitation period and the similarity of the personal goal, but only one of these may be determined, or the similarity may not be determined in either case. That is, the similarity score calculation unit 111 may calculate the similarity on the basis of any item included in the information about the reference patient and the target patient.

The similarity ranking determination unit 112 corresponds to the similarity ranking determination unit 12 in FIG. 1, and ranks the reference patients in descending order of similarity scores.

The first rearrangement processing unit 113 corresponds to the first rearrangement processing unit 13 in FIG. 1, and rearranges the rank of the reference patients so that the reference patients including more rehabilitation menus commonly performed on a plurality of reference patients among the rehabilitation menus operated on the reference patients are ranked higher. More specifically, the first rearrangement processing unit 113 refers to the rehabilitation history storage unit 102, and acquires the history of the rehabilitation menu provided to the reference patient ranked by the similarity ranking determination unit 112. Then, the first rearrangement processing unit 113 extracts a rehabilitation menu commonly operated on a plurality of reference patients among the acquired rehabilitation menus. Then, the number of reference patients operated for each extracted rehabilitation menu is calculated, and the rehabilitation menus are ranked in a descending order of the number of reference patients who have been operated. Thereafter, the first rearrangement processing unit 113 performs rearrangement processing of changing the ranking set by the similarity ranking determination unit 112 so that the ranking of the reference patients including the rehabilitation menu for which a plurality of reference patients have been operated becomes higher.

For example, in a case where the number of reference patients who have been operated by the first rehabilitation menu is five and the number of reference patients who have been operated by the second rehabilitation menu is three, the first rearrangement processing unit 113 ranks the reference patients including the first rehabilitation menu to be high regardless of the similarity score, and ranks the reference patients including the second rehabilitation menu next to the patients including the first rehabilitation menu. In addition, the reference patient including the first rehabilitation menu and the second rehabilitation menu is ranked to be high among the reference patients including the first rehabilitation menu.

The output control unit 103 generates a list of reference patients in the rankings rearranged by the first rearrangement processing unit 113, and displays the list as a similar patient list on the terminal apparatus 500. Furthermore, the list includes the rehabilitation menu for each reference patient read by the first rearrangement processing unit 113 as information related to each reference patient. In addition, the rehabilitation menu for each reference patient preferably includes all of the rehabilitation menus operated on the patient. Furthermore, the list preferably includes past information of the reference patient and a recovery history by rehabilitation.

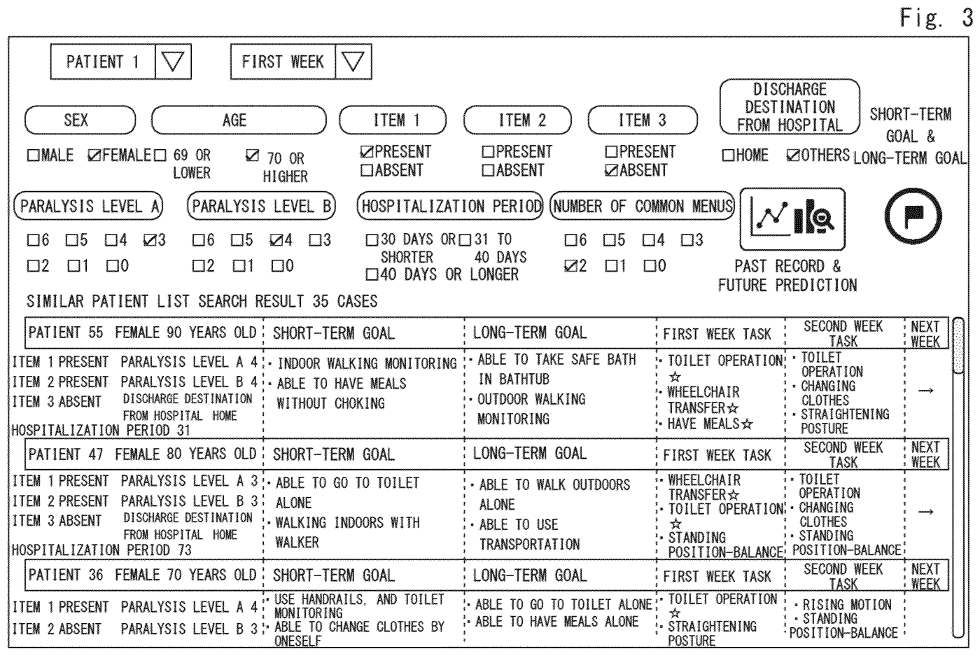

Here, FIG. 3 is a schematic diagram illustrating an example of a user interface screen displayed on the terminal apparatus 500 by the output control unit 103. In the example illustrated in FIG. 3, there is an interface for inputting information about the target patient on an upper side of the screen (a portion from the left in the drawing), and a similar patient list in which the reference patients are rearranged by the similarity ranking determination unit 112 and the first rearrangement processing unit 113 is displayed on a lower side of the screen (a portion from the right in the drawing).

As the information of the target patient, it is preferable that information substantially equivalent to items included in the past information of the reference patient such as attribute information such as a sex and an age, and an item indicating the ability level of the patient can be input. In addition, it is also possible to exclude an item that is not a search target of a similar patient among a plurality of pieces of target patient information from search items by not checking a check box of the item.

The target patient may be selected from patients registered as patient information recorded in the past information storage unit 101, and for example, in this case, the screen is configured in a state where a check box such as attribute information related to the target patient is input by selecting a patient.

The input item is provided with a check box for selecting the number of common menus. The first rearrangement processing unit 113 sets a program upper limit number designated by a user with this check box as an upper limit of the number of types of rehabilitation menus to be verified as being commonly operated.

Furthermore, with regard to the list of similar patients, a reference patient having past information similar to that of the target patient has a high similarity score, and a reference patient including a rehabilitation menu having a large number of operated reference patients is displayed higher. In addition, as items related to each reference patient, information input items of the target patient and a rehabilitation menu provided to each reference patient are displayed in a horizontal direction in time series in which the rehabilitation menu is performed.

In addition, with regard to the common rehabilitation menu among the rehabilitation menus of the reference patient, if there is an easy-to-understand display such as “asterisk” as illustrated in the example of FIG. 3, the visibility of the user can be enhanced.

By referring to such a list of similar patients, the user (for example, a therapist) can efficiently refer to the rehabilitation menu operated on many patients among the rehabilitation menus operated on patients having similar characteristics and the degree of recovery obtained by the rehabilitation menu. Then, the user can easily determine the effect obtained by the recommended rehabilitation menu recommended by another system with reference to such information.

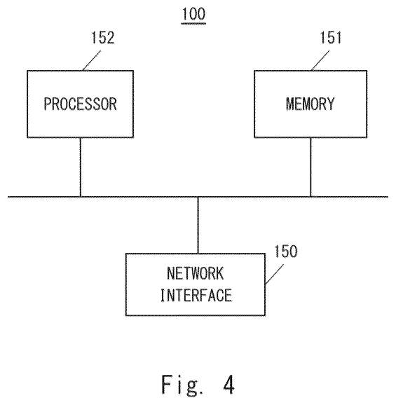

FIG. 4 is a schematic view illustrating an example of a hardware configuration of the rehabilitation menu determination assistance apparatus 100. As illustrated in FIG. 4, the rehabilitation menu determination assistance apparatus 100 includes a network interface 150, a memory 151, and a processor 152.

The network interface 150 is used to communicate with any other apparatus such as the terminal apparatus 500. The memory 151 includes, for example, a combination of a volatile memory and a nonvolatile memory. The memory 151 is used for storing software (a computer program) including one or more commands to be executed by the processor 152, data used for various processes of the rehabilitation menu determination assistance apparatus 100, and the like. The past information storage unit 101 and the rehabilitation history storage unit 102 illustrated in FIG. 2 are implemented by, for example, the memory 151, but these may be implemented by other storage devices.

The processor 152 reads and executes software (computer program) from the memory 151 to perform processing of the patient information acquisition unit 110, the similarity score calculation unit 111, the similarity ranking determination unit 112, the first rearrangement processing unit 113, and the output control unit 103 illustrated in FIG. 2. The processor 152 may be, for example, a microprocessor, a micro processor unit (MPU), a central processing unit (CPU), or the like. The processor 152 may include a plurality of processors.

As described above, the rehabilitation menu determination assistance apparatus 100 has a function as a computer. Note that, similarly, the terminal apparatus 500 has a hardware configuration as illustrated in FIG. 4. That is, the processing of the terminal apparatus 500 is realized, for example, by execution of a program by a processor.

In addition, the program described above may be stored by using various types of non-transitory computer-readable media to be supplied to a computer. The non-transitory computer-readable media include various types of tangible storage media. Examples of non-transitory computer-readable medium include a magnetic recording medium (for example, a flexible disk, a magnetic tape, or a hard disk drive), a magneto-optical recording medium (for example, a magneto-optical disc), a CD-read only memory (ROM) CD-R, a CD-R/W, and a semiconductor memory (for example, a mask ROM, a programmable ROM (PROM), an erasable PROM (EPROM), a flash ROM, and a random access memory (RAM)). In addition, the program may be supplied to a computer through various types of transitory computer-readable media. Examples of transitory computer-readable media include electrical signals, optical signals, and electromagnetic waves. The transitory computer-readable medium can provide the program to the computer via a wired communication line such as an electric wire and optical fibers or a wireless communication line.

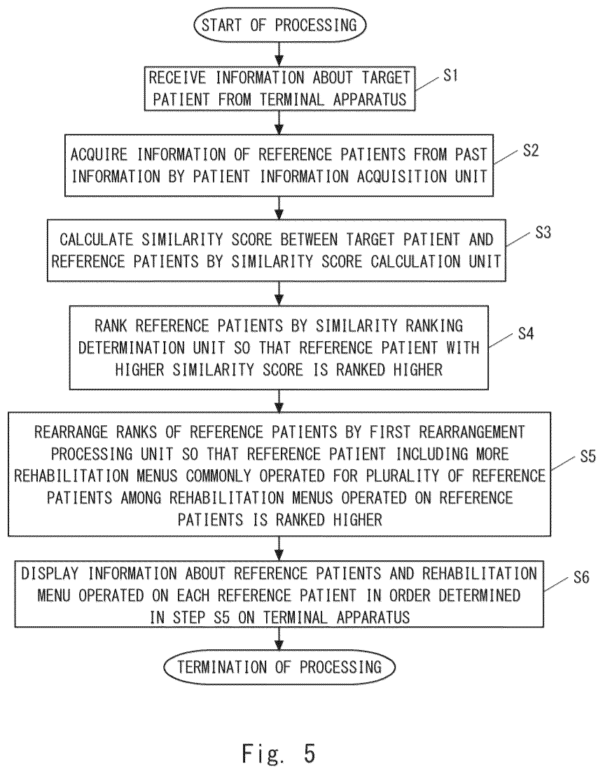

Next, a flow of an operation of the rehabilitation menu determination assistance apparatus 100 will be described. FIG. 5 is a flowchart illustrating an example of an operation of the rehabilitation menu determination assistance apparatus 100. Hereinafter, an operation flow will be described with reference to FIG. 5.

As illustrated in FIG. 5, first, the rehabilitation menu determination assistance apparatus 100 receives information about the target patient from the terminal apparatus 500 (step S1). Next, in the rehabilitation menu determination assistance apparatus 100, the patient information acquisition unit 110 refers to the past information storage unit 101 to acquire information of a reference patient for reference of the rehabilitation menu to be operated on the target patient received in step S1 (step S2). Next, in the rehabilitation menu determination assistance apparatus 100, the similarity score calculation unit 111 calculates a similarity score between the target patient and the reference patient for each reference patient (step S3). Next, in the rehabilitation menu determination assistance apparatus 100, the similarity ranking determination unit 112 ranks the reference patients in a descending order of the similarity score (step S4). Next, in the rehabilitation menu determination assistance apparatus 100, the first rearrangement processing unit 113 rearranges the ranking of the reference patients so that the reference patients including more rehabilitation menus commonly performed for a plurality of reference patients among the rehabilitation menus operated on the reference patient are ranked higher (step S5). Next, the rehabilitation menu determination assistance apparatus 100 displays the information about the reference patient and the rehabilitation menu operated on each reference patient in the order determined in step S5 on the terminal apparatus 500 (step S6).

The rehabilitation menu determination assistance apparatus 1 according to the first example embodiment has been described above. According to the present system, it is possible to preferentially refer to a rehabilitation menu having many operated patients among rehabilitation menus operated on a reference patient similar to the target patient among other patients other than the target patient. As a result, the user (for example, a therapist) can efficiently refer to the progress of the target patient when the recommended rehabilitation menu recommended by another system is applied, and can easily determine whether or not to apply the recommended rehabilitation menu.

### Second Example Embodiment

In a second example embodiment, a rehabilitation menu determination assistance apparatus 2 that is another form of the rehabilitation menu determination assistance apparatus 1 according to the first example embodiment will be described. Note that, in the description of the second example embodiment, the same constituent elements as those in the first example embodiment are denoted by the same reference numerals as those in the first example embodiment, and the description thereof will be omitted.

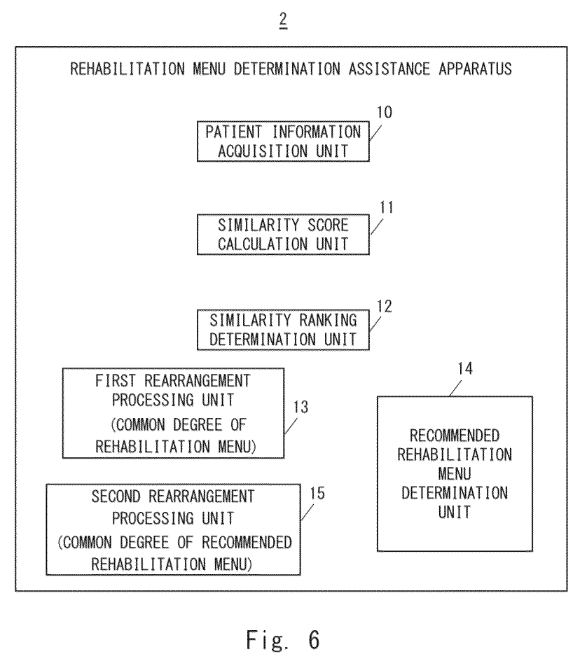

FIG. 6 is a block diagram illustrating an example of a rehabilitation menu determination assistance apparatus according to an outline of the second example embodiment. As illustrated in FIG. 6, the rehabilitation menu determination assistance apparatus 2 is obtained by adding a recommended rehabilitation menu determination unit 14 and a second rearrangement processing unit 15 to the rehabilitation menu determination assistance apparatus 1. In the second example embodiment, the reference patient rank rearranged by the first rearrangement processing unit 13 is further rearranged according to the presence or absence of the recommended rehabilitation menu in consideration of the degree of recovery of the reference patient and the like.

The recommended rehabilitation menu determination unit 14 determines a rehabilitation menu having a record of maximizing the recovery amount among the rehabilitation menus operated on the reference patient as a recommended rehabilitation menu to be determined as the recommended rehabilitation menu.

The second rearrangement processing unit 15 rearranges the ranks of the reference patients so that the reference patients including more recommended rehabilitation menus are ranked higher. More specifically, the second rearrangement processing unit 15 performs a rearrangement process on the basis of the rank of the reference patient subjected to the rearrangement by the first rearrangement processing unit 13.

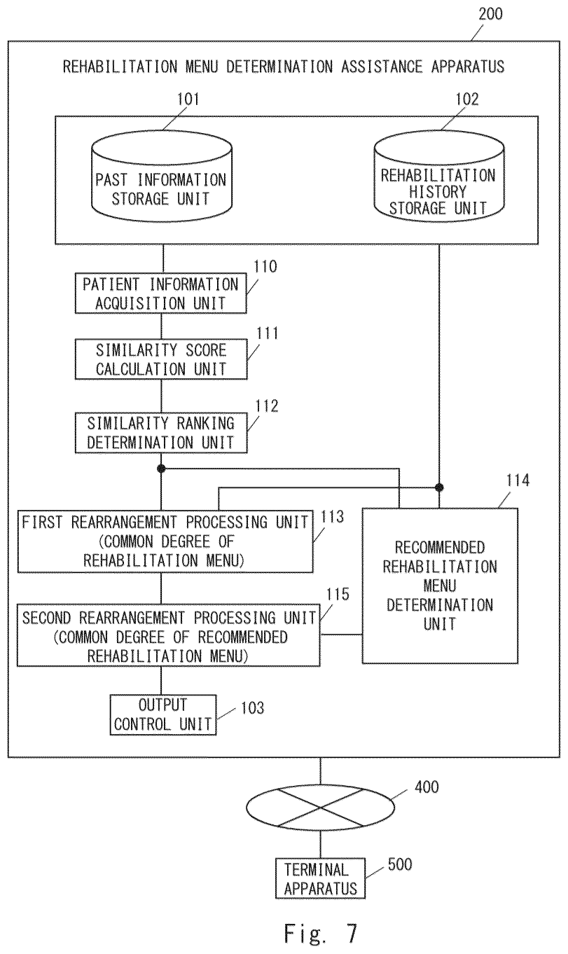

FIG. 7 is a block diagram illustrating an example of a configuration of a rehabilitation menu determination assistance system according to the second example embodiment. As illustrated in FIG. 7, a rehabilitation menu determination assistance apparatus 200 can realize the function of the rehabilitation menu determination assistance apparatus 2 by a server or a computer similarly to the rehabilitation menu determination assistance apparatus 100.

The recommended rehabilitation menu determination unit 114 corresponds to the recommended rehabilitation menu determination unit 14 in FIG. 6 and determines a rehabilitation menu having a record of maximizing the recovery amount among the rehabilitation menus operated on the reference patients as a recommended rehabilitation menu to be determined as the recommended rehabilitation menu. More specifically, the recommended rehabilitation menu determination unit 114 acquires the rehabilitation history of the reference patients ranked by the similarity ranking determination unit 112 from the rehabilitation history storage unit 102. The past information of the rehabilitation history storage unit 102 includes the ability values of the reference patient before and after the rehabilitation indicated by the rehabilitation history. Then, the recommended rehabilitation menu determination unit 114 sets the rehabilitation menu in which the degree of improvement in the ability value of the reference patient after execution of the rehabilitation indicated by the rehabilitation history is maximized as the recommended rehabilitation menu, that is, the recommended rehabilitation menu determination unit 114 sorts the rehabilitation menus in a descending order of the recovery degree on the basis of the degree of improvement in the ability value of the reference patient after execution of the rehabilitation indicated by the rehabilitation history. The recommended rehabilitation menu determination unit 114 performs sorting in accordance with how much the ability value has been recovered by the reference patient similar to the target patient performing rehabilitation indicated by the rehabilitation history, and determines a rehabilitation menu with a high degree of recovery as a recommended rehabilitation menu.

The second rearrangement processing unit 115 corresponds to the second rearrangement processing unit 15 in FIG. 6, and rearranges the ranks of the reference patients so that the reference patients including more recommended rehabilitation menus are ranked higher. More specifically, the second rearrangement processing unit 115 performs rearrangement such that reference patients including a large number of rehabilitation menus indicated as recommended rehabilitation menus are ranked high on the basis of the rank of reference patients subjected to rearrangement by the first rearrangement processing unit 13.

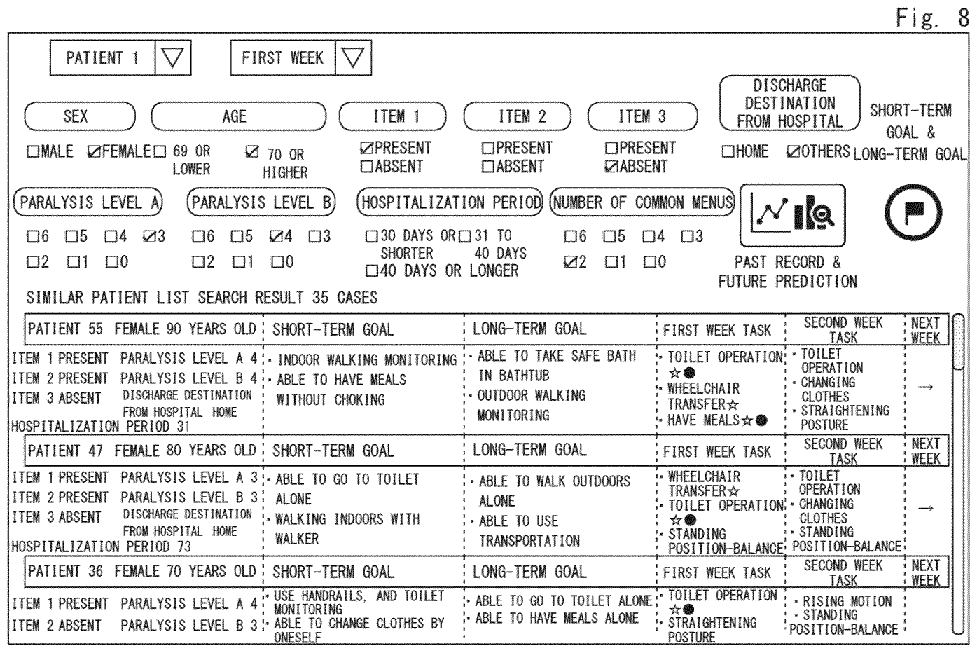

FIG. 8 is a schematic view illustrating an example of a user interface screen displayed on a terminal apparatus in the rehabilitation menu determination assistance system according to the second example embodiment. The example illustrated in FIG. 8 is basically the same as the interface screen illustrated in FIG. 3, but in the second example embodiment, The recommended rehabilitation menu operated on the reference patient to be rearranged by the second rearrangement processing unit 115 includes display different from the common menu, for example, “circle”. By performing such display, the visibility of the user can be enhanced.

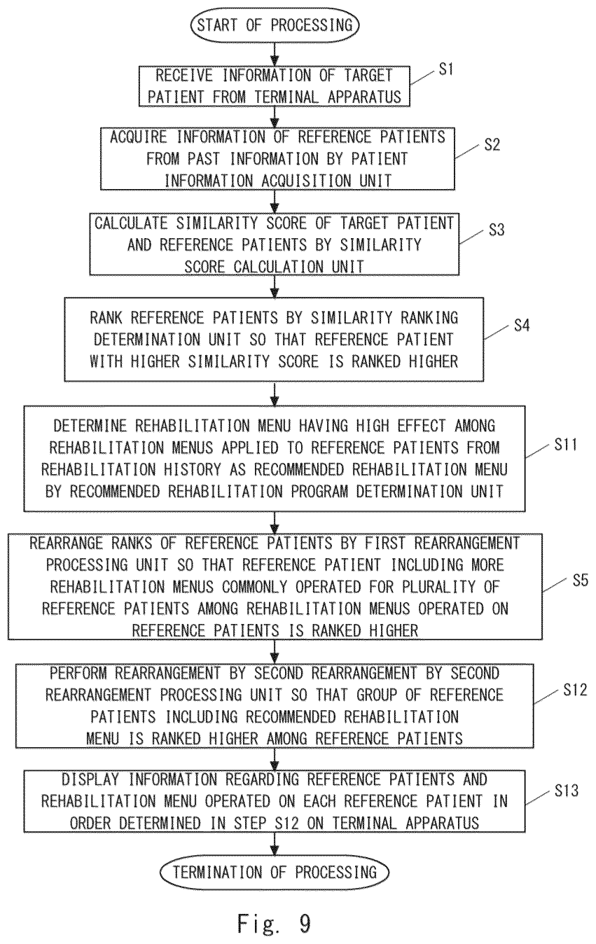

FIG. 9 is a flowchart illustrating an example of an operation of the rehabilitation menu determination assistance apparatus 200 according to the second example embodiment. As illustrated in FIG. 9, first, the rehabilitation menu determination assistance apparatus 200 receives information about the target patient from the terminal apparatus 500 (step S1). Next, in the rehabilitation menu determination assistance apparatus 200, the patient information acquisition unit 110 refers to the past information storage unit 101 to acquire information of a reference patient similar to the patient information received in step S1 (step S2). Next, in the rehabilitation menu determination assistance apparatus 200, the similarity score calculation unit 111 calculates a similarity score between the target patient and the reference patient for each reference patient (step S3). Next, in the rehabilitation menu determination assistance apparatus 200, the similarity ranking determination unit 112 ranks the reference patients in a descending order of the similarity score (step S4). Next, in the rehabilitation menu determination assistance apparatus 200, the recommended rehabilitation menu determination unit 114 determines, as a recommended rehabilitation menu, a rehabilitation menu having a high effect (having a high degree of recovery) among the rehabilitation menus applied to the reference patients from the rehabilitation history storage unit 102 (step S11). Next, in the rehabilitation menu determination assistance apparatus 200, the first rearrangement processing unit 113 rearranges the ranking of the reference patients so that the reference patients including more rehabilitation menus commonly performed for a plurality of reference patients among the rehabilitation menus operated on the reference patient are ranked higher (step S5). Next, in the rehabilitation menu determination assistance apparatus 200, the second rearrangement processing unit 115 performs the rearrangement so that a group of reference patients including the recommended rehabilitation menu is ranked higher among the reference patients (step S12). Next, the rehabilitation menu determination assistance apparatus 200 displays the information regarding the reference patients and the rehabilitation menu operated on each reference patient in the order determined in step S512 on the terminal apparatus 500 (step S13).

The rehabilitation menu determination assistance apparatus 2 according to the second example embodiment has been described above. According to the present system, among other patients other than the target patient, it is possible to preferentially refer to a reference patient having a large number of common rehabilitation menus and further including a large number of recommended rehabilitation menus. As a result, the user (for example, a therapist) can efficiently refer to the progress of the target patient when the recommended rehabilitation menu recommended by another system is applied, and can more easily determine whether or not to apply the recommended rehabilitation menu as compared with the first example embodiment.

Although the invention of the present application has been described above with reference to the example embodiments, the invention of the present application is not limited to the above. Various modifications that can be understood by those skilled in the art can be made to the configuration and details of the invention of the present application within the scope of the invention.

This application claims priority based on Japanese Patent Application No. 2022-10530 filed Jan. 26, 2022, the entire disclosure of which is incorporated herein.

### REFERENCE SIGNS LIST

1, 2 REHABILITATION MENU DETERMINATION ASSISTANCE APPARATUS
10 PATIENT INFORMATION ACQUISITION UNIT
11 SIMILARITY SCORE CALCULATION UNIT
12 SIMILARITY RANKING DETERMINATION UNIT
13 FIRST REARRANGEMENT PROCESSING UNIT
14 RECOMMENDED REHABILITATION MENU DETERMINATION UNIT
15 SECOND REARRANGEMENT PROCESSING UNIT
100 REHABILITATION MENU DETERMINATION ASSISTANCE APPARATUS
101 PAST INFORMATION STORAGE UNIT
102 REHABILITATION HISTORY STORAGE UNIT
103 OUTPUT CONTROL UNIT
110 PATIENT INFORMATION ACQUISITION UNIT
111 SIMILARITY SCORE CALCULATION UNIT
112 SIMILARITY RANKING DETERMINATION UNIT
113 FIRST REARRANGEMENT PROCESSING UNIT
114 RECOMMENDED REHABILITATION MENU DETERMINATION UNIT
115 SECOND REARRANGEMENT PROCESSING UNIT
150 NETWORK INTERFACE
151 MEMORY
152 PROCESSOR
200 REHABILITATION MENU DETERMINATION ASSISTANCE APPARATUS
400 NETWORK
500 TERMINAL APPARATUS

What is claimed is:

1. A rehabilitation menu determination assistance apparatus comprising:
At least one memory; and
At least one processor coupled to the at least one memory and configure to execute the instructions to:
acquire reference patients having individual information from past information;
calculate a similarity score indicating similarity of individual information between an input target patient and the reference patients;
rank the reference patients in a descending order of the similarity score; and
rearrange ranks of the reference patients so that the reference patients including more rehabilitation menus commonly performed for a plurality of the reference patients among rehabilitation menus operated on the reference patients are ranked higher.

2. The rehabilitation menu determination assistance apparatus according to claim 1, wherein the at least one processor is further configured to execute the instructions to:
determine as a recommended rehabilitation menu, the rehabilitation menu having a record of maximizing a recovery amount among the rehabilitation menus operated on the reference patients; and
rearrange the reference patients so that the reference patients including more recommended rehabilitation menus are ranked higher.

3. The rehabilitation menu determination assistance apparatus according to claim 1, wherein at least one processor coupled to the at least one memory and configure to execute the instructions to:
set a program upper limit number designated by a user as an upper limit of the number of types of rehabilitation menus to be verified as being commonly operated.

4. The rehabilitation menu determination assistance apparatus according to claim 1, wherein at least one processor coupled to the at least one memory and configure to execute the instructions to:
calculate the similarity score by using an item designated as an item for which similarity determination is performed by a user as a calculation target of the similarity score.

5. The rehabilitation menu determination assistance apparatus according to claim 1, wherein
the individual information of the target patient and the individual information of the reference patients include a history of ability values during a predetermined rehabilitation period, and
wherein at least one processor coupled to the at least one memory and configure to execute the instructions to:
calculate at least a similarity of a history of the ability values.

6. The rehabilitation menu determination assistance apparatus according to claim 1, wherein
the individual information of the target patient and the individual information of the reference patients include goals in rehabilitation, and
wherein at least one processor coupled to the at least one memory and configure to execute the instructions to:
calculate at least the similarity of the goals.

7. The rehabilitation menu determination assistance apparatus according to claim 2, wherein
the past information includes ability values of the reference patients before and after execution of rehabilitation indicated by a rehabilitation history of the reference patients, and
wherein at least one processor coupled to the at least one memory and configure to execute the instructions to:
set as the recommended rehabilitation menu, the rehabilitation menu in which an improvement degree of an ability value of the reference patients after execution of rehabilitation indicated by the rehabilitation history is maximized.

8. (canceled)

9. A rehabilitation menu determination assistance method executed by arithmetic processing of a computer system including a storage device storing past information, the method comprising:
patient information acquisition processing of acquiring reference patients having individual information from past information;
similarity score calculation processing of calculating a similarity score indicating similarity of individual information between an input reference patient and the reference patients;
similarity ranking determination of ranking the reference patients in a descending order of the similarity score; and
first rearrangement processing of rearranging ranks of the reference patients so that the reference patients including more rehabilitation menus commonly performed for a plurality of the reference patients among rehabilitation menus operated on the reference patients are ranked higher.

10. A non-transitory computer-readable medium storing a rehabilitation menu determination assistance program causing a computer to execute:
patient information acquisition processing of acquiring reference patients having individual information from past information;
similarity score calculation processing of calculating a similarity score indicating similarity of individual information between an input reference patient and the reference patients;
similarity ranking determination processing of ranking the reference patients in a descending order of the similarity score; and
first rearrangement processing of rearranging ranks of the reference patients so that the reference patients including more rehabilitation menus commonly performed for a plurality of the reference patients among rehabilitation menus operated on the reference patients are ranked higher.
